(19) United States
(12) Patent Application Publication
(10) Pub. No.: US 2015/0148591 A1
(43) Pub. Date: May 28, 2015

(54) INCONTINENCE IMPLANT, INSERTION DEVICE AND RELATED METHODS OF USE

(71) Applicant: Boston Scientific Scimed, Inc., Maple Grove, MN (US)

(72) Inventors: Sharmad S. Joshi, Natick, MA (US); Michael S.H. Chu, Brookline, MA (US); John Goncalves, New Bedford, MA (US)

(21) Appl. No.: 14/549,169

(22) Filed: Nov. 20, 2014

Related U.S. Application Data
(63) Continuation of application No. 61/909,653, filed on Nov. 27, 2013.

(51) Int. Cl. A61F 2/00

(52) U.S. Cl. CPC A61F 2/0063; A61F 2/0045; A61F 2002/0072
USPC 600/37

(57) ABSTRACT

The present invention discloses medical systems and related methods of use. One such medical system includes an implant assembly and an insertion device. The insertion device includes an elongate member and a hub member. The elongate member includes a needle portion and a handle portion. The hub member is movably coupled to the handle portion of the elongate member. The hub member is configured to be removably coupled to the implant assembly.

### CROSS-REFERENCE TO RELATED APPLICATION

This application is a Nonprovisional of, and claims priority to, U.S. Patent Application No. 61/909,653, filed on Nov. 27, 2013, entitled “INCONTINENCE IMPLANT, INSERTION DEVICE AND RELATED METHODS OF USE”, the disclosure of which is incorporated by reference herein in its entirety.

### BACKGROUND

1. Field

The present invention generally relates to surgical devices and procedures, particularly devices and methods for the delivery of implants within a patient's body.

2. Description of the Related Art

Anatomical tissues such as pelvic tissues may be weakened or damaged with age, injury, or disease. This decrease in structural integrity of anatomical tissues may have significant medical consequences, which in turn might influence the biological functions of the tissues. There are various surgical procedures for treating such dysfunction of the tissues. Implants can be placed into a patient to provide support for the weakened or damaged tissue. The support provided by the implant may replicate the natural position and structure of the tissue, and thereby help in decreasing or eliminating impairment of biological functions resulting from tissue weakening or damage.

Surgical methods may use an insertion device to deliver the implant at the anatomical tissue inside the patient's body. Such an insertion device assists in the delivery and placement of the implant. The insertion device may include a needle coupled directly with the implant or through a dilator. The dilator passes through tissues that have already been pierced by the needle, thereby dilating a needle track for ease of the implant introduction and positioning within the patient. A curved and small diameter needle may be used to minimize injury to the patient.

In view of the above, there is a need for an insertion device and a surgical procedure that reduces or prevents bladder injury or injury to other portions of the body of the patient and provides desired needle stiffness or sharpness. In some embodiments, the device allows the operator to better control the needle trajectory and prevent inadvertent or harmful exposure of the needle, such as a tissue piercing portion of the needle, to tissue within a patient's body.

### SUMMARY

In an embodiment, the present invention discloses an insertion device, a medical system and a method for delivery of an implant inside a patient's body. The insertion device includes an elongate member and a hub or connector member. The elongate member includes a needle portion and a handle portion. The hub or connector member is movably coupled to the handle portion of the elongate member. The hub member is configured to be removably coupled to an implant assembly.

In another embodiment, a system includes an implant assembly and an insertion device. The insertion device includes an elongate member and a hub member. The elongate member includes a needle portion and a handle portion. The hub member is movably coupled to the handle portion of the elongate member. The hub member is also configured to be removably coupled to the implant assembly.

In yet another embodiment, a method is disclosed. The method includes inserting a medical device into a body of a patient to a first location within the body of the patient. The medical device includes an implant assembly and an insertion device. The insertion device includes an elongate member and a hub member. The elongate member includes a needle portion and a handle portion. The hub member is movably coupled to the handle portion of the elongate member. The hub member is configured to be removably coupled to the implant assembly. The method further includes moving the hub member from a first position with respect to the elongate member to a second position with respect to the elongate member. Moreover, the method includes moving the medical device from the first location within the body of the patient to a second location within the body of the patient.

### BRIEF DESCRIPTION OF THE FIGURES

The invention and the following detailed description of certain embodiments thereof may be understood with reference to the following figures:

FIG. 1 is a schematic illustration of a medical system, according to an embodiment of the invention.

FIG. 2 is a perspective view of an insertion device, according to an embodiment of the invention.

FIG. 3 is a perspective view of a medical system, according to an embodiment of the invention.

FIG. 4 is a side view of a portion of the medical system of FIG. 3 including a tubular member, according to an embodiment of the invention.

FIG. 5 is a perspective view of the insertion device of FIG. 3 in a first configuration.

FIG. 6 is a perspective view of the insertion device of FIG. 3 in a second configuration.

FIG. 7 is a top view of an implant assembly, according to an embodiment of the invention.

FIG. 8 is a side view of a portion of a medical system, according to an embodiment of the invention.

FIGS. 9 and 10 are top views of portions of the implant assembly of FIG. 8.

FIGS. 11-15 are side views of distal portions of insertion devices, according to embodiments of the invention.

FIG. 16 is a schematic illustration of a medical system being inserted into a body of a patient.

FIGS. 17 and 18 schematically illustrate sling members disposed within a patient's body.

FIG. 19 is a flow chart of a method for delivery of a medical system into a patient's body, according to an embodiment of the invention.

### DETAILED DESCRIPTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting, but rather to provide an understandable description of the invention.

The terms “a” or “an,” as used herein, are defined as one or more than one. The term “another,” as used herein, is defined as at least a second or more. The terms “including” and/or “having”, as used herein, are defined as comprising (i.e., open transition).

The terms proximal and distal described in relation to various medical devices, apparatuses, and components as discussed in the subsequent text of the present invention are referred with a point of reference. The point of reference, as used in this description, is a perspective of an operator. The operator may be a surgeon, a physician, a nurse, a doctor, a technician, and the like, who may perform the procedure or surgery through the patient's body orifice or incision as described in the present invention. The term proximal refers to an area that is closer to the operator. The term distal refers to an area that is farther from the operator. The patient, as referred to here, can be a human female, male or any other mammal.

Although the present invention focuses on devices, systems and methods for use primarily for treatment of urinary incontinence, the disclosed devices, systems and methods may be used to treat any type of pelvic floor disorder or in any other procedure to place an implant within a body of a patient. In addition, the devices and methods may be used for delivering diagnostic or treatment devices to other parts of a patient's body with minimal damage to surrounding tissue. The present invention discloses devices that allow the operator to have greater control over the trajectory of a needle disposed on an insertion device to avoid inadvertent damage to surrounding tissue, such as the bladder of the patient.

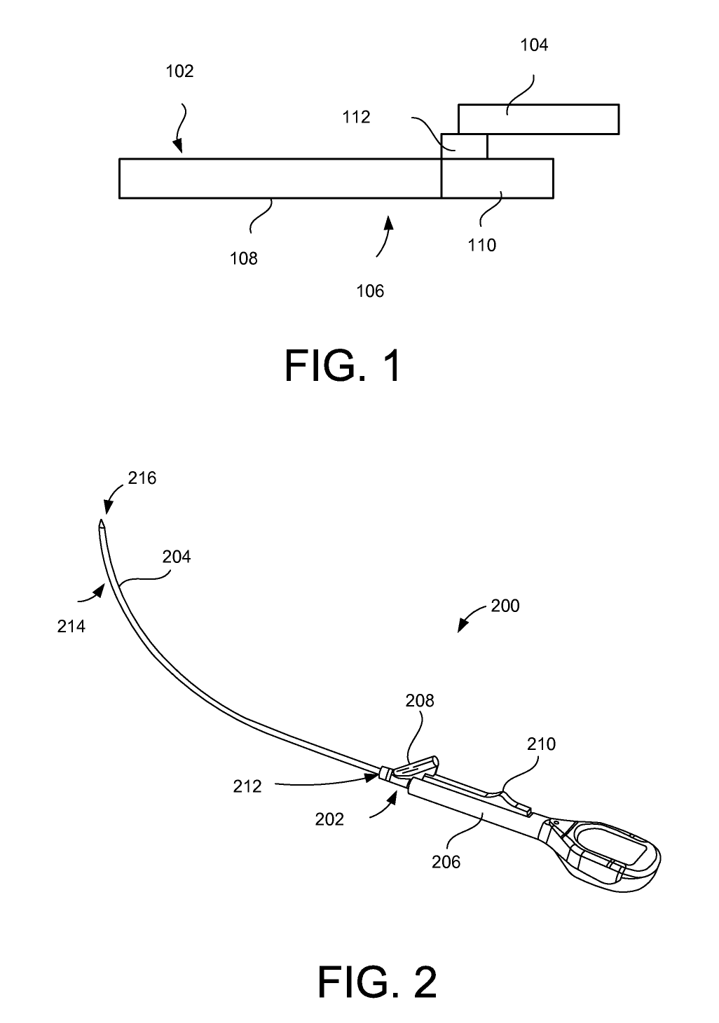

FIG. 1 is a schematic illustration of a medical system or device 100 configured to treat a pelvic floor disorder. The medical system 100 includes an insertion device 102 and an implant assembly 104 for delivery and implantation at a target location inside a patient's body. The implant assembly 104 is coupled to the insertion device 102. In certain embodiments, the insertion device 102 can be designed so as to be used in the trans-vaginal procedure. Other embodiments may include insertion device 102 that may be adapted to deliver the implant assembly 104 either laproscopically or through other bodily openings or bodily incisions

As shown, the insertion device 102 includes an elongate member 106, and a hub or connector member 112 that is movably coupled to the elongate member 106. The elongate member 106 includes a needle portion 108 coupled to a handle portion 110. An operator can control the trajectory of a distal needle portion 108 via a proximal handle portion 110. The hub member 112 is movably coupled to the handle portion 110 of the elongate member 106. In some embodiments, the hub member 112 can be configured to move with respect to the handle portion 110 and various such movements can be, but not limited to, rotation, pivoting, sliding or a combination of these. In certain embodiments, the hub member 112 can be configured to move the implant assembly 104 and/or a portion of the implant assembly 104 with respect to the needle portion 108.

The implant assembly 104 is removably coupled to the hub member 112 of the insertion device 102, i.e., the implant assembly 104 can be detached from the insertion device 102 after implantation or during an implantation procedure. The hub or connection member 112 may be any type of connection or coupling member that is configured to be removably coupled to the implant assembly 104. Before the procedure, the implant assembly 104 can be attached to the insertion device 102 and detached after the implant assembly 104 reaches the treatment location or at another time during an implantation procedure.

In some embodiments, the needle portion 108 is fixedly coupled to the handle portion 110 of the elongate member 106. For example, the connection between the needle portion 108 and the handle portion 110 can be permanent and can be accomplished through threading, chemical bonding, heat molding, gluing, tight-fitting, fastening or any such mechanisms. In some embodiments, the needle portion 108 can be unitarily formed or monolithically formed with the handle portion 110. In certain other embodiments, the needle portion 108 can be detachably and/or adjustably coupled to the handle portion 110 using a screw mount, bayonet mount, or a similar detachable system. The needle portion 108 can be configured to pierce through tissue layers during the medical procedure. For this purpose, the needle portion 108 can include a sharp needle tip (such as a tissue piercing tip) at its distal end. The needle portion 108 is a substantially rigid, rod-like structure and in some examples, the needle portion 108 can have a curved configuration along at least a portion of its length. Other variations may also be contemplated. In some embodiments, the needle portion 108 can have a substantially constant diameter along its length, or can have a variable diameter along all or a portion of its length. The diameter and length and curvature of the needle portion 108 can vary based on the surgical requirements. In certain embodiments, the needle portion 108 can be a surgical needle with a substantially small outer diameter for minimally invasive surgery. In some embodiments, needle portion 108 can be formed of a stainless steel material (e.g., surgical grade stainless steel).

In an embodiment, the needle portion 108 is coupled to the handle portion 110 such that the needle portion 108 is adapted to be introduced within a patient's body and the handle portion 110 remains external to the patient's body. An operator may manipulate the handle portion 110 to introduce and drive the needle portion 108 into the patient's body. The handle portion 110 can be ergonomically designed to allow the operator to easily hold and control the insertion device 102. In certain embodiments, the handle portion 110 can have various configuration such as regular, irregular, smooth, curved or any configuration that facilitates handling of the insertion device 102. The handle portion 110 can be manufactured from a rigid or flexible plastic material, or a combination thereof. Exemplary plastic materials include polycarbonate, lexan, Acrylonitrile butadiene styrene (ABS), and the like without limitations.

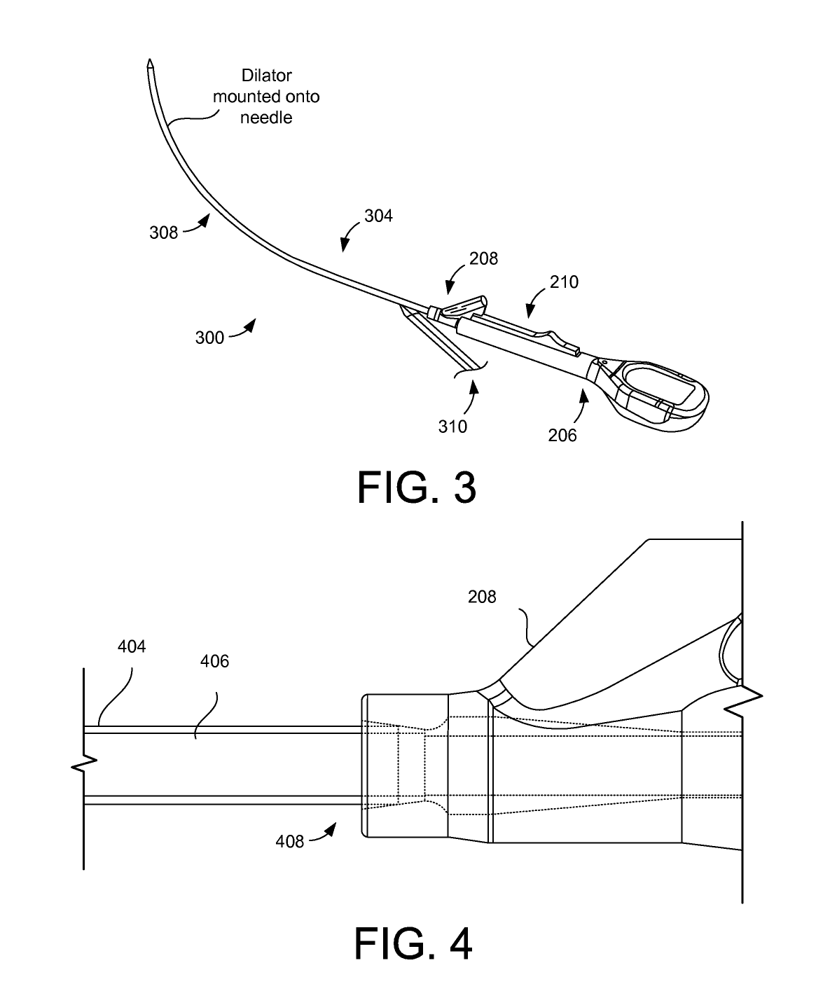

In certain embodiments, the medical device 100 includes a tubular member (see FIG. 3) such as a dilator that can be moved to cover the tip of the needle portion 108 in order to prevent the tip of the needle portion 108 from piercing unintended portions (such as bladder) of the patient's body. The tubular member can be moved to uncover the tip of the needle portion 108 for piercing through the desired tissue layers. In this manner, the medical device 100 allows the operator use the device 100 with the tip of the needle portion 108 uncovered when piercing through tough bodily tissue or when moving the device within the body of the patient at locations distant from sensitive areas (such as the bladder). The operator may then use the device with the tip of the needle portion 108 covered and move the device within the body of the patient near sensitive areas (such as the bladder) so as to avoid any unintended puncturing of the sensitive areas (such as the bladder). For example, in one embodiment, the operator of the medical device 100 may insert the device 100 though a vaginal incision with the device 100 in a first configuration (with the tip of the needle portion 108 exposed or uncovered). Once the device 100 nears or begins passing by the bladder of the patient, the operator may place the device 100 in a second configuration (for example, the operator may move the tubular member such that it covers the tip of the needle portion). In this configuration, the device 100 can be moved though tissue proximate the bladder (such as through the space of Retzius) with less chance of inadvertently piercing the bladder (as the tip of the needle portion is covered by the tubular member). Once the device 100 has passed through the tissue proximate the bladder, the operator may move the device back to the first configuration and may pass the device through additional tissue, such as the abdominal wall.

FIG. 2 is a perspective view of an insertion device 200, according to one embodiment of the invention. The insertion device 200 includes an elongate member 202 having a needle portion 204 coupled to a handle portion 206. A hub member 208 is movably coupled to the handle portion 206 of the elongate member 202 such that hub member 208 may be moved from a first position to a second position. In some examples, the hub member 208 may be rotated with respect to the elongate member 202. In other examples, the hub member 208 may be pivoted about the elongate member 202. In FIG. 2, the hub member 208 may slide along the elongate member 202 without limitations.

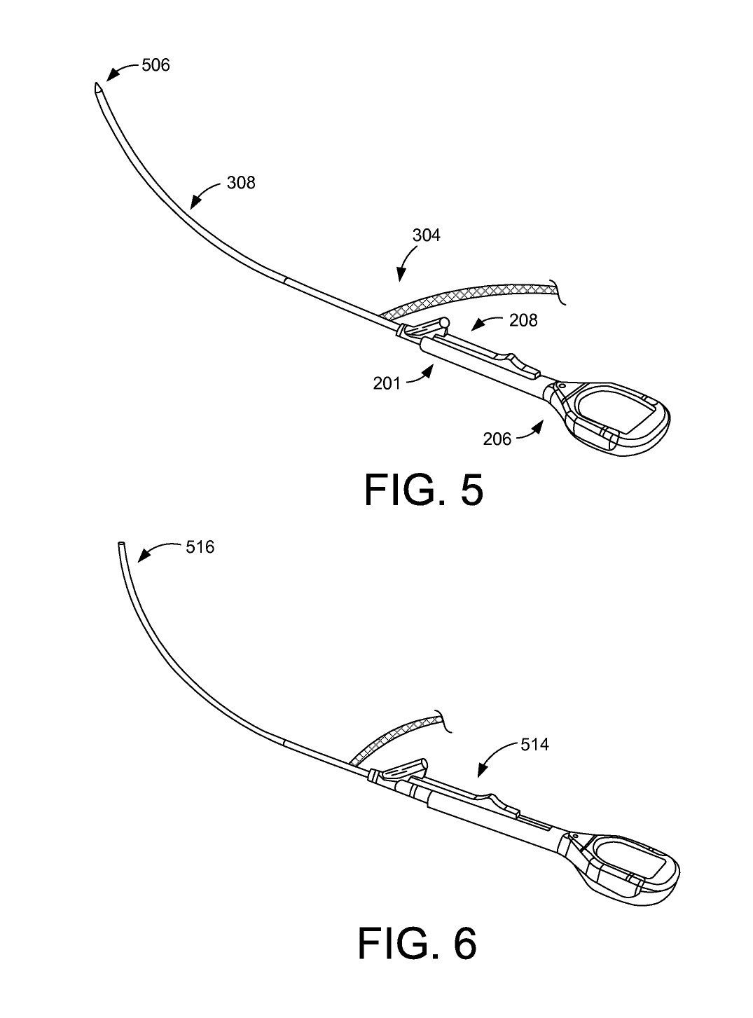

As shown, the hub member 208 can include an actuator 210 such as a slider that is configured to move the hub member 208 with respect to the elongate member 202 from a first position to a second position. Thus, the operator can move the slider 210 distally to move the hub member 208 from a first position to a second position (as shown in FIGS. 5 and 6). In other embodiments, the actuator 210 can include gears, push buttons, or any other type of actuator facilitating movement of the hub member 208 with respect to the elongate member 202.

In some embodiments, the hub member 208 may be formed monolithically with the slider 210. For example, the hub member 208 and the slider 210 are both formed from a single piece of material. In certain other embodiments, the hub member 208 may be formed separately from the slider 210 and then coupled to the slider 210. For example, the hub member 208 and slider 210 can be coupled in an abutting relationship, an overlapping relationship, or can be bridged. The hub member 208 can be coupled to the slider 210 by, for example, heat bonding, gluing, using fasteners, sewing or any other methods of coupling known in the art.

The needle portion 204 has a proximal end 212 and a distal end 214. The proximal end 212 is coupled to the handle portion 206 of the elongate member 202, while the distal end 214 includes a tip 216 for piercing tissue. In FIG. 2, the proximal end 212 of the needle portion 204 is coupled to the handle portion 206 such that the needle portion 204 passes through the lumen of the hub member 208. The tissue piercing tip 216 can have various shapes such as curved, cone, circular or the like without limitations. In some embodiments, the tissue piercing tip 216 may be sharp and configured to dissect tissue layers and create a passageway within bodily tissues to deliver and place the implant assembly inside the patient's body. In some embodiments, the needle portion 204 can be made of stainless steel or other medical grade metal. In an embodiment, the needle portion 204 can be a surgical needle with a substantially small outer diameter for minimally invasive surgery.

In some embodiments, the needle portion 204 is fixedly coupled to the handle portion 206 of the elongate member 202. For example, the connection between the needle portion 204 and the handle portion 206 can be permanent and can be accomplished through threading, chemical bonding, heat molding, gluing, tight-fitting, fastening or any such mechanisms. In certain other embodiments, the needle portion 204 can be detachably and/or adjustably coupled to the handle portion 206 using a screw mount, bayonet mount, or a similar detachable system. The needle portion 204 is a substantially rigid, rod-like structure and in some examples, the needle portion 204 can have a curved configuration along at least a portion of its length. Other configurations may also be contemplated. In some embodiments, the needle portion 204 can have a substantially constant diameter along its length, or can have a variable diameter along all or a portion of its length. The diameter and length and curvature of the needle portion 204 can vary based on the surgical requirements. In certain embodiments, the needle portion 204 can be a surgical needle with a substantially small outer diameter for minimally invasive surgery.

FIG. 3 is a perspective view of a medical system 300 including a tubular member, according to an embodiment of the invention. The medical system 300 includes an insertion device 201, which is similar in function and structure to the insertion devices (102, 200) discussed in FIGS. 1 and 2 respectively. An implant assembly 304 can be coupled to the insertion device 201. The implant assembly 304 can be, for example, an incontinence implant or sling (such as a retropubic incontinence implant) or any other implant (such as an implant to help correct a prolapse or a hernia) configured to be delivered by way of a transvaginal approach or a transobturator approach or vaginal pre-pubic approach or can be delivered through other approaches and positioned at various locations within a patient's body. In certain embodiments, the implant assembly 304 can be coupled to a hub member 306 during a portion of the medical procedure such as implantation procedure and can be detached after the implant assembly 304 reaches the treatment location or at another time during an implantation procedure.

The implant assembly 304 includes a tubular member 308 and a sling member 310 coupled to the tubular member 308. The sling member 310 can be configured to be coupled to the tubular member 308 such that the sling member 310 can be implanted at a target location inside a patient's body.

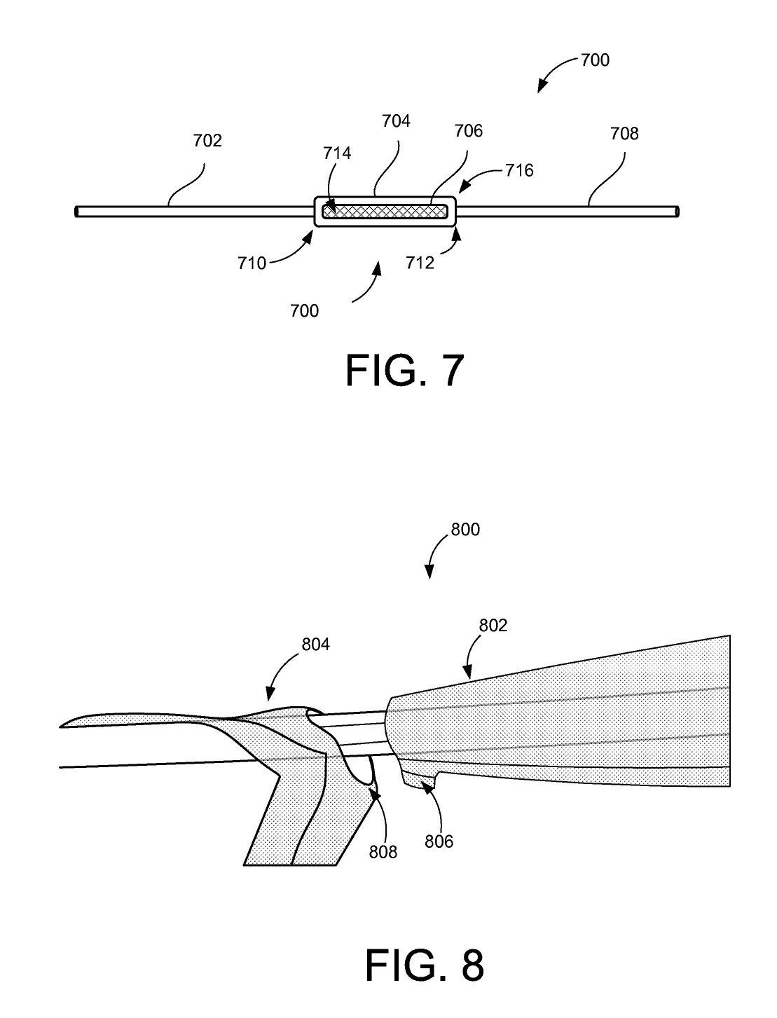

In some embodiments, the tubular member 308 can be a dilator. In some embodiments, the sling member 310 can be an implant, for example, a mesh. In some embodiments, the mesh 310 can be coupled to an outer surface of the dilator 308. In other embodiments, the mesh 310 can be coupled to an inside surface of the tubular member or dilator 308. In additional embodiments, the mesh 310 can be coupled to a sleeve and then the sleeve can be coupled to the dilator 308, and this will be discussed in more detail with reference to FIG. 7. In some embodiments, the mesh 310 can be made of a polymeric material that may include a natural and/or a synthetic material. Exemplary synthetic polymeric materials are polypropylene, polyester, polyethylene, nylon, PVC, polystyrene, and the like.

In some embodiments, the dilator 308 can be coupled to the mesh 310 through a suture. In an exemplary embodiment, the suture can form an association loop to couple the dilator 308 with the mesh 310. Various other coupling mechanisms may be used, such as, for example, an adhesive, a staple, a fastener, or thread can be used to couple the dilator 308 with the mesh 310.

In some embodiments, the tubular member 308 in the form of a dilator can be configured to expand an opening in a patient's body allowing insertion of the mesh and an insertion device 201 as they follow the dilator 308 into the opening within a patient's body. In other words, the dilator 308 can be used to assist the delivery of the mesh 310 to the pelvic region or other location within the body of the patient. The dilator 308 defines a lumen and can be slid onto a needle portion toward a handle portion 206 such that the dilator 308 reaches the hub member 208, thereby allowing coupling of the implant assembly 304 with the insertion device 201. Upon sliding or placing the dilator 308 over the needle portion, at least a portion of the needle portion of the elongate member is disposed within a passageway/lumen defined by the dilator 308 when the implant assembly 304 is coupled to the hub member 306. At least in part because of the flexible nature of the dilator material, the dilator 308 can be sized to assume the shape of the needle portion on its insertion. In an embodiment, the length of the dilator 308 can be longer than a length of the needle portion or can be smaller than the length of the needle portion depending on a particular application.

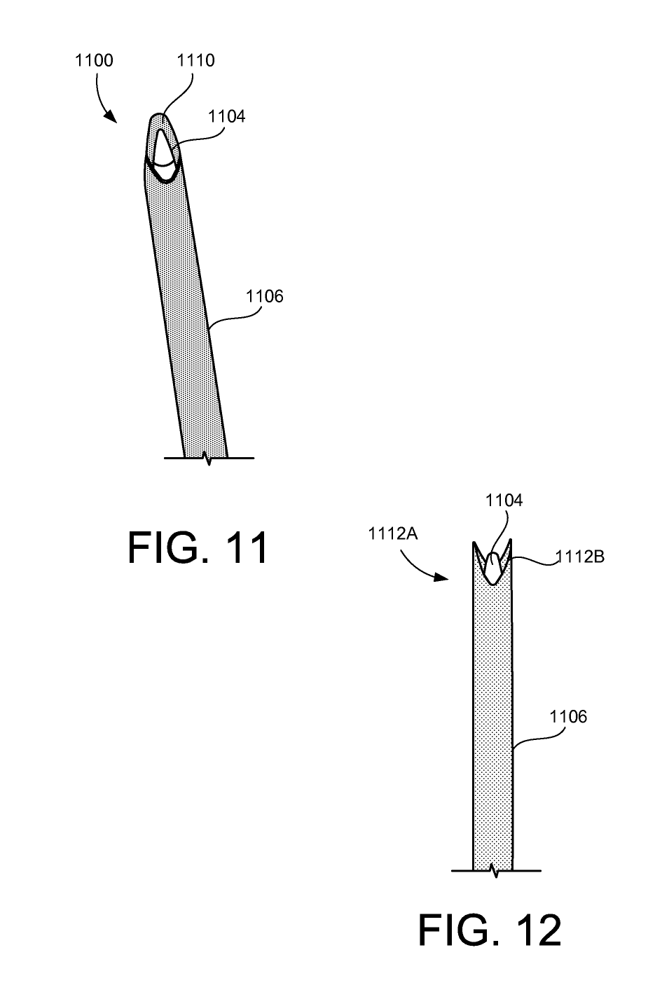

The dilator 308 can have a variety of lengths, shapes, diameters, etc. In some embodiments, the dilator 308 can be made from one or more biocompatible materials such as a plastic or metal. In an example, the dilator 308 can be made of a semi-rigid plastic material. Various examples of such materials include, but are not limited to, polyethylene terephthalate (PET), polyethylene (PE), or ethylene vinyl acetate (EVA). In some embodiments, the cross section of the dilator 308 can be circular, substantially flat or triangular in shape or can be of any other shape. In other embodiments, the cross section of the dilator 308 can be substantially rectangular and tapered at the distal portion such that the tip of the needle portion protrudes out to contact the tissue. Various configurations of the dilator 308 will be discussed below with reference to FIGS. 11-15. In some embodiments, the tip or end portion of the dilator 308 may be configured to be in an open or closed position. For example, the tip may be in an open position to allow the tip of the needle portion to protrude out of the dilator and may be in a closed position when the tip of the needle portion is not protruding or extending from the dilator. In some embodiments, as shown and discussed in more detail below, the dilator may define a slit or slot or a plurality of slits or slots.

In some embodiments, the dilator 308 includes a first end portion, and a second end portion opposite to the first end portion. The first end portion is coupled to the mesh 310, whereas the second end portion includes a flared tip (See FIGS. 11-15). In an embodiment, the dilator 308 is a first dilator that can be attached to a first end portion of the mesh 310. In an embodiment, a second dilator that can be similar to the dilator 308 and can be attached to a second end portion of the mesh 310.

FIG. 4 is a side view of a portion of the medical system of FIG. 3 including a hub member 208, a dilator 404 and a needle portion 406. Specifically, FIG. 4 shows a coupling mechanism between the hub member 208 and the dilator 404 that could be used in some embodiments. To this end, the hub member 208 includes a cavity 408 or recess that is configured to receive the dilator 404. In some embodiments, the cavity 408 or recess is configured to frictionally couple the dilator 404 to the hub member 208. For example, the dilator 404 can be press-fit to the hub member 208. Upon insertion, the dilator 404 passes over the needle portion 406. In additional embodiments, the dilator 404 can be rigidly coupled to the hub member 208. In accordance with several other embodiments, various other types of engagement mechanism can be employed, such as, but not limited to, snap fitting, thread fitting, bolt jointing, riveting, and the like. In some embodiments, the cavity 408 can be configured to have various shapes, lengths, and widths to house the dilator 404.

FIG. 5 is a perspective view of the medical system 300 of FIG. 3 in a first position. The medical system 300 includes an insertion device 201, and an implant assembly 304 coupled to the insertion device 201. The insertion device 201 is configured to be coupled to the implant assembly 304 such that the implant assembly 304 can be delivered to the pelvic region of the patient's body. The insertion device 201 includes a needle portion, a handle portion 206 and a hub member 208 movably coupled to the handle portion 206. In the first position, the hub member 208 is configured to be deployed in a first position, i.e., a resting position such that a tissue piercing tip 506 of the needle portion is disposed outside of a lumen (See FIG. 6) defined by the dilator 308 (or extends from the distal end portion of the dilator 308). In other words, the tissue piercing tip 506 of the needle portion remains uncovered or exposed to the outside environment such as the bodily tissue when inserted. In this position, the needle tip 506 is disposed out of the lumen defined by the dilator 308 and can pierce through tissue layers.

FIG. 6 is a perspective view of the medical system of FIG. 3 in a second position. An operator may activate an actuator 514 on the hub member 208 from the first position to a second position. This motion can move the dilator 308 such that the tissue piercing tip 506 of the needle portion is covered by the distal end of the dilator 308. Thus, the tip 506 remains inside the lumen 516 of the dilator 308 to avoid any tissue disruption or perforation when navigating through a patient's body or when passing through sensitive areas, such as, for example, near the bladder wall. As shown in FIG. 6, the operator may move the hub member 208 with respect to the elongate member using the actuator 514 to the second position. For example, pushing or sliding the actuator 514 forward can move the dilator 308 distally to cover the tip 506 of the needle portion. Using this mechanism, the operator can cover the needle tip 506 when it is near sensitive tissue (such as the bladder wall) and when a sharp tip is not needed to advance the insertion device 201 (such as when it is traversing through space of Retzius). In some embodiments, the tip or end portion of the dilator may be configured to be placed in an open configuration (when the tip of the needle portion extends through the tip or end portion of the dilator) and a closed configuration (when the tip of the needle portion does not extend from the dilator and is disposed within the dilator).

In contrast, pulling back the actuator 514 moves the dilator 308 back to a first position (shown in FIG. 5) to uncover the tip 506 of the needle portion for piercing tissue layers. In this manner, the operator can reduce or prevent unnecessary damage to sensitive tissues such as the bladder wall. By sliding mechanism of the hub member 208 (i.e., the actuator 514), the operator can expose the tip 506 of the insertion device 201 only when it is needed, thereby helping to avoid unnecessarily damage to surrounding tissues.

FIG. 7 is a top view of an implant assembly 700, according to an embodiment of the invention. The implant assembly 700 includes a first dilator 702, a sleeve 704, an implant 706, and a second dilator 708. The sleeve 704 can be used to cover a portion of the implant 706 protecting the implant 706 from damage during delivery and preventing premature engagement of the implant 706 to surrounding tissue. In some embodiments, the implant 706 is delivered to the pelvic region of the patient through one or more vaginal incisions, and/or through exterior incisions in the patient. In some embodiments, the sleeve 704 can be transparent, semi-transparent, colored, non-colored, or a combination of these. In some embodiments, the sleeve 704 can have various configurations such as tapered, flat, tubular or others. The sleeve 704 can be formed from a flexible biocompatible polymer, and can be configured to allow the operator to view the implant 706 disposed within the sleeve 704. In some embodiments, the sleeve 704 may extend along a partial length or a full length of the implant 706.

In some embodiments, the implant 706 can be coupled to the dilator (such as 702, 708) through the sleeve 702. For example, the implant 706 is first coupled to the sleeve 704 and then the sleeve 704 is coupled to the dilator 702, 708. In an embodiment, the first dilator 702 can be attached to a first end portion 710 of the sleeve 704. In an embodiment, the second dilator 708 that can be similar to the first dilator 702 can be attached to a second end portion 712 of the sleeve 704. The dilator 702, 708 can be coupled to the sleeve 704 through various means such as crimping, heat sealing, stitching, stretching, tip tipping, etc. In certain other embodiments, the dilator 702, 708, can be directly coupled to the implant 706 without using the sleeve 704. For example, the first dilator 702 can be coupled to a first end portion 714 of the implant 706, and the second dilator 708 can be coupled to a second end portion 716 of the implant 706.

FIG. 8 is a side view of a portion of a medical system 800 according to an embodiment of the invention. The medical system 800 shows a portion of an insertion device such as a hub member 802 and a portion of an implant assembly such as a tubular member 804 (for example, a dilator). FIG. 8 shows the coupling mechanism between the hub member 802 and the dilator 804. The hub member 802 is shown to include a projection member 806 such as a tab. In an embodiment, the projection member 806 can be defined by an extended portion or a separately attached portion at a proximal portion of the hub member 802. The dilator 804 includes an aperture 808 to match the shape and size of the projection member 806. The aperture 808 can be formed of various shapes and sizes. Exemplary shapes can be a rectangle, a square, or circular, elliptical, and slotted, and an oval shape.

The projection member 806 is configured to extend into or through the aperture 808 defined by the dilator 804 to couple the dilator 804 to the hub member 802. In some embodiments, the aperture 808 can be sized to frictionally retain the hub member 802, thereby forming a connection between the hub member 802 and the dilator 804. In some embodiments, the projection member 806 can be configured to be releasably mate with the aperture 808. In certain other embodiments, the projection member 806 can be snap fit into the aperture 808 to secure and lock the dilator 804 with respect to the hub member 802.

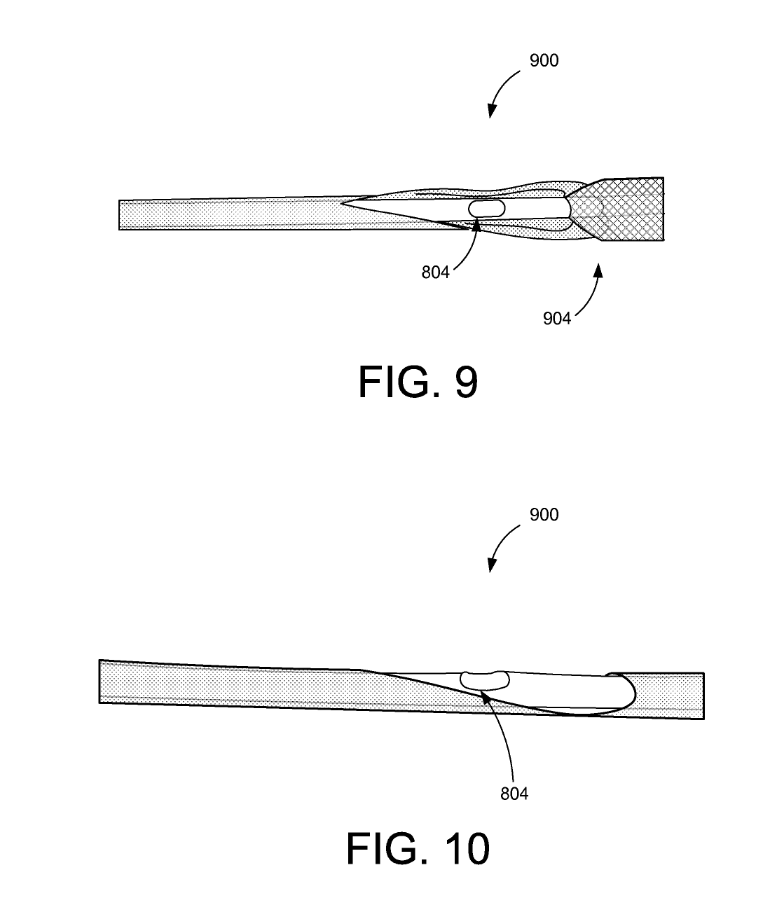

FIGS. 9 and 10 are side top views of portions of an implant assembly 900 according to an embodiment of the invention. Specifically, FIGS. 9 and 10 show closeup views of the implant assembly of FIG. 8. The implant assembly 900 includes a tubular member 804, and a sling member 904 coupled to the tubular member 804. The tubular member 804 includes a sidewall defining a lumen, the sidewall defines an opening 906. The projection member defined by the hub member 802 can be configured to extend through the opening 906 defined by the sidewall of the tubular member 804 to removably couple the implant assembly 900 to the hub member 802. When the implant assembly 900 is coupled to the hub member 802, at least a portion of the needle portion is disposed within the lumen defined by the tubular member 804. In some embodiments, the implant assembly 900 can be coupled

In some embodiments, the opening 906 can be formed of various shapes and sizes. Exemplary shapes can be a rectangle, a square, or circular, elliptical, and slotted, and an oval shape. In some embodiments, the opening 906 or a portion of the opening 906 may be formed of a flexible material to help create a tight fit. For example, in some embodiments, the opening 906 may include a ring of flexible material (such as an “0” ring).

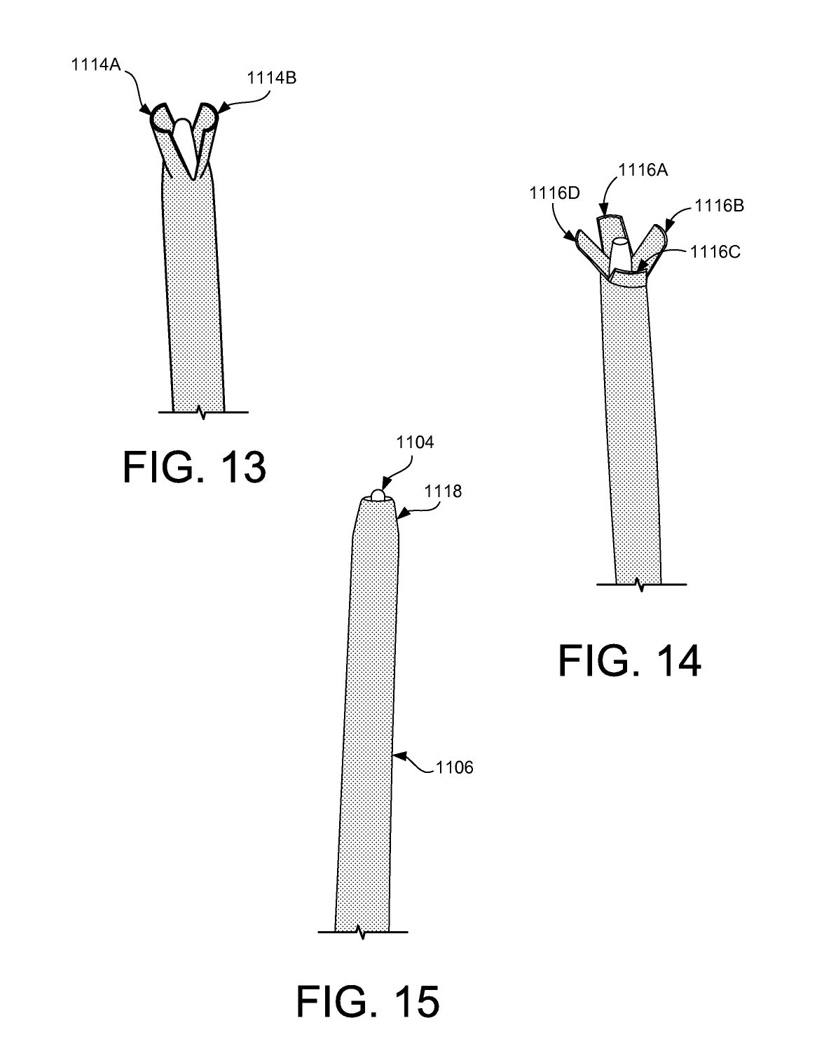

FIGS. 11-15 are side views of portions of medical devices, according to embodiments of the invention. The medical device 1100 includes a needle portion having a tissue piercing tip 1104, and a dilator 1106 having a tip. Different configurations of the dilator tip are contemplated (for example, as shown in FIGS. 11-15) for improving navigation with minimal damage to surrounding tissue. In FIG. 11, the dilator tip is configured to have a tapered shape with one tab 1110, which may help navigate through tissue easily and minimize damage to surrounding tissue. FIG. 12 shows a dilator tip with two tapering tabs 1112a, 1112b. In FIGS. 13 and 14, the dilator tip has been cut into two tabs 1114a, 1114b, or four tabs 1116a, 1116b, 1116c, 1116d respectively and they are directed outwardly away from the tip 1104 of the needle portion. In FIG. 15, dilator 1106 is configured to have a curved or tapered shape (marked as 1118). This configuration may facilitate navigation inside the patient's body. Additionally, in some embodiments, the configuration may facilitate the opening and closing of the tip of the dilator.

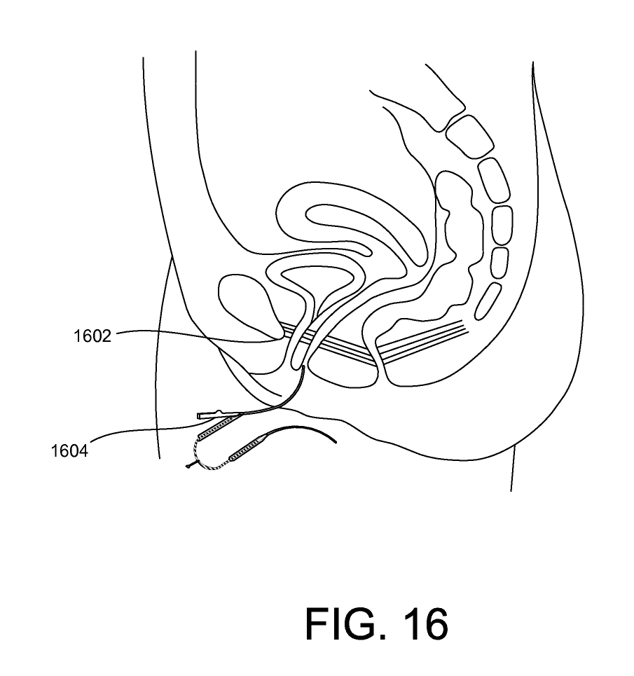

FIG. 16 is a schematic illustration of medical device 1604 being inserted into a body of a patient. FIG. 16 illustrates positioning of the medical device 1604 inside a female body through a vaginal approach. A vaginal incision in the vaginal space 1602 (such as an anterior vaginal incision) can be made and the medical device 1604 can be positioned.

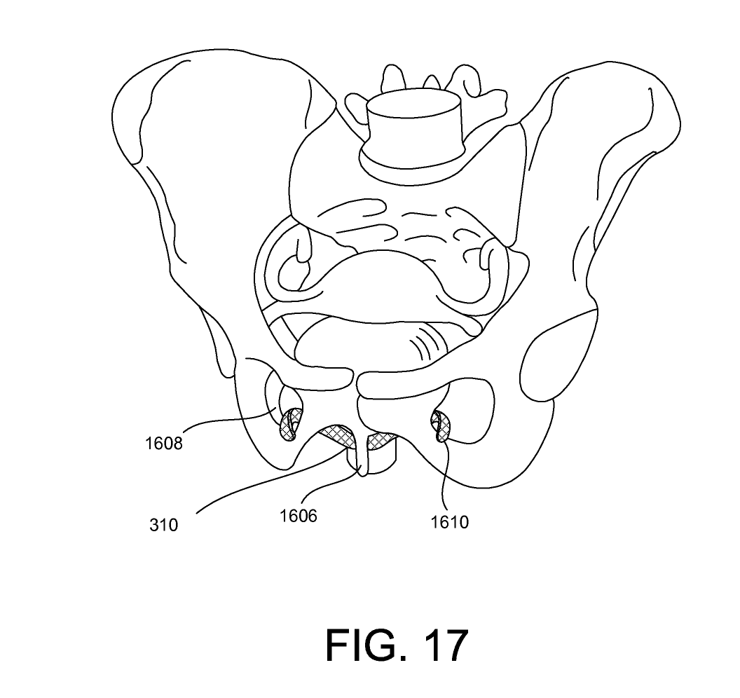

FIG. 17 illustrates implantation of the implant 310 inside a female body through a transobturator approach. In accordance with this approach, the implant 310 is passed under the urethra 1606 and out through incisions in a groin compartment of the thigh (not shown in the diagram). A first end portion of the implant 310 can be attached at the first portion 1608 within the pelvic floor region. A second end portion of the implant 310 can be attached at the second portion 1610 within the pelvic floor region. In an embodiment, the first end portion 1608 and the second end portion 1610 can be the obturator foreman tissues proximate the obturator foreman.

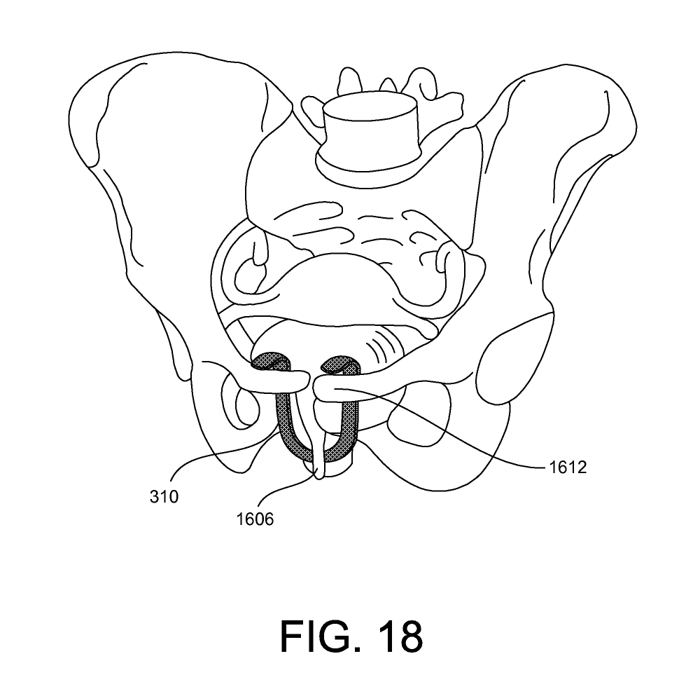

FIG. 18 illustrates implantation of the implant 310 inside the female body through a retropubic approach. The retropubic approach positions the implant 310 under the urethra 1606 in a U shape. The first end portion and the second end portion of the implant 310 are brought up behind a pubic bone 1612 and out through skin incisions above the pubic bone, and coupled to tissues adjacent to the pubic bone 1612.

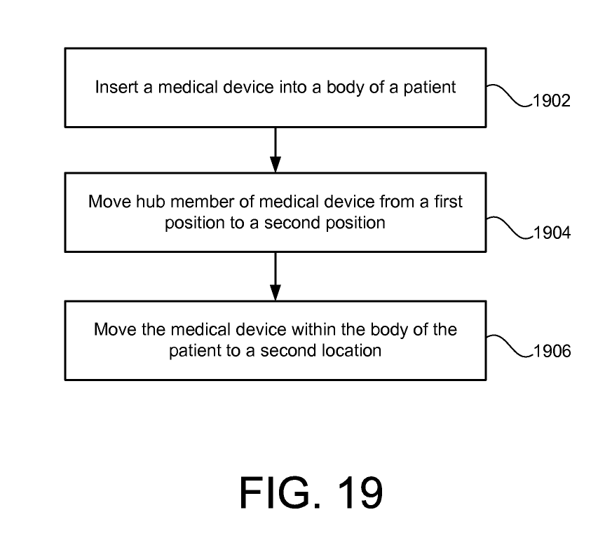

FIG. 19 is a flow chart of a method for delivery of a medical device/system such as the medical device in a patient's body for treatment of a pelvic floor disorder, in accordance with an embodiment of the present invention. In an embodiment, the pelvic floor region can be a retropubic region. Referring to FIG. 19, in conjunction with FIG. 3, the method of delivery and placement of the bodily implant with the use of the medical device 300 is described in accordance with an embodiment of the present invention. The medical device 300 is hereafter used to describe the method in an exemplary embodiment. However, it must be appreciated that other medical devices such as the medical devices of FIGS. 1 and 2 may also be employed in a similar manner and these medical devices (100, 200) have already been described in conjunction with various figures above.

The method includes inserting the medical device 300 such as the insertion device 201 along with the dilator 308 into a body of a patient, at 1902. In an embodiment, the method may include creating a vaginal incision for delivery of the insertion device 201 and the dilator 308 within the body through a trans-vaginal approach. In some embodiments, the vaginal incision can be created using devices such as knife, scalpel, or the like. The medical system 300 includes the insertion device 201 and an implant assembly 304 coupled to the insertion device 201. The insertion device 201 includes the needle portion coupled to the handle portion 206 and the hub member 208 movably coupled to the handle portion 206 of the elongate member. In an embodiment, the method can include assembling the dilator 308 and the hub member 208 before the delivery into the patient's body such that the assembling includes sliding the projection member into the aperture sized to frictionally retain the projection member and lock the dilator 308 with the insertion device 201, particularly, with the hub member 208. In an embodiment, the medical system 300 can be pre-assembled and the operator may not be required to perform the step of assembling.

When inserting the medical device 300, the hub member 208 is configured to be in a first position such that the tip of the needle portion is disposed out of the lumen defined by the dilator 308 to pierce through a tissue layer. Then, medical device 300 is passed through the tissue layer to expand an opening in the patient's body and navigated such that the medical device 300 reaches a first location within the patient's body. The method includes positioning of the implant 310 at the first location of the pelvic floor region. For example, the first location can be a location near the bladder (such as in the space of Retzius). After reaching the first location, at 1904 the hub member 208 is moved to a second position such that the dilator 308 covers the tip of the needle portion and the medical device is navigated through the patient's body to reach to second location within the patient's body at 1906. The tip of the needle portion is covered so that needle tip does not cause any harm to the sensitive tissue layers, such as the bladder wall. Here, the second location can be a location away from the first location. For example, the second location can be near their abdominal wall of the patient. When the device reaches the second location the hub member can be moved back to the first position such that the tip of the needle is exposed. The device, with the needle tip exposed can then be passed through the abdominal wall and exit the body of the patient.

In some embodiments, the method includes dis-associating or removing the dilator 308 from the hub member 208 once the insertion device 201 extends from the body of the patient (such as out the abdominal wall or other skin incision). For example, the dilator can be removed from the hub and needle and the needle can be removed from the body by backing the needle out the vaginal incision. The dilator can then be removed from the implant and pulled from the body via the abdominal skin incision leaving the implant within the body of the patient.

In an embodiment, another insertion device (or the same insertion device) similar to the insertion device 201 can be coupled to the second dilator in a manner similar to that described above. In another embodiment, the insertion device 201 can be coupled to the second dilator to implant 310 the second end portion to the target location. In some embodiments, the implant forms a sling around or below (to provide support to) a portion of the body of the patient. For example, the implant can form a sling and support a urethra of the patient.

In some embodiments, the method further includes removing the dilator 308 from the patient's body after disassociating the dilator 308 from the hub member 208 once the insertion device 201 reaches the target location in the pelvic floor region. The operator also disassociates the sleeve by cutting a suture, wherein the suture couples the sleeve with the dilator such as the first dilator. In an embodiment, the first dilator and the sleeve can be removed from the implant 310. The sleeve and the first dilator can then be pulled out from the patient's body through incisions such as provided in an abdomen or in a groin or an obturator area of the patient. The second dilator can also be removed in a similar manner. In some embodiments, the method includes removing the insertion device 201 from the patient's body after fixing the implant 310 or before tensioning or fixing the implant 310.

In some embodiments, the dilator 308 is coupled to the implant 310 and the method includes attaching a first end portion of the implant 310 at the first portion within the pelvic floor region 1608. In some embodiments, the method includes attaching a second end portion of the implant 310 at a second portion within the pelvic floor region 1608. In some embodiments, the method includes removing the insertion device 201 from the patient's body after fixing the implant 310.

Upon implanting the implant 310 at the target location, the method further includes fixing the bodily implant at a first portion within the pelvic floor region 1608. In an embodiment, the pelvic floor region 1608 is a retropubic region. The method further includes attaching the first end portion and the second end portion of the implant 310, respectively, at the first portion and the second portion within the pelvic floor region 1608 as described in FIG. 17.

In some embodiments, the medial device as described above, may be used in portions or locations within the body other than the pelvic region of the patient. For example, the medial device may be used to insert and implant and provide support to any portion of a body of a patient. The implant may be any type of bodily implant. For example, the implant may be any type of implant that is configured to provide support to the body of the patient. The implant may be a mesh sling that is configured to provide support to a portion of a body of a patient. In some embodiments, the implant may be configured to treat or help prevent pelvic organ prolapses or hernias.

In some embodiments, an insertion device includes an elongate member having a needle portion and a handle portion; and a hub member movably coupled to the handle portion of the elongate member, the hub member being configured to be removably coupled to an implant assembly.

In some embodiments, the hub member is slideably coupled to the elongate member. In some embodiments, the needle portion of the elongate member is fixedly coupled to the handle portion of the elongate member. In some embodiments, the hub member defines a cavity that is configured to receive at least a portion of the implant assembly to frictionally couple the implant assembly to the hub member. In some embodiments, the hub member includes a projection member, the projection member being configured to extend through a portion of the implant assembly to couple the implant assembly to the hub member.

In some embodiments, a system includes an implant assembly; and an insertion device, the insertion device having an elongate member and a hub member, the elongate member having a needle portion and a handle portion, the hub member movably coupled to the handle portion of the elongate member, the hub member being configured to be removably coupled to the implant assembly.

In some embodiments, the implant assembly includes a tubular member and a sling member, the sling member being coupled to the tubular member.

In some embodiments, the implant assembly includes a tubular member and a sling member, the tubular member defining a lumen, at least a portion of the needle portion of the elongate member being configured to be disposed within the lumen defined by the tubular member when the implant assembly is coupled to the hub member.

In some embodiments, the implant assembly includes a tubular member and a sling member, the tubular member defining a lumen, the hub member defining a cavity, at least a portion of the implant assembly being disposed within the cavity defined by the hub member to couple the implant assembly to the hub member.

In some embodiments, the implant assembly includes a tubular member and a sling member, the tubular member defining a lumen, the hub member defining a cavity, at least a portion of the implant assembly being disposed within the cavity defined by the hub member to couple the implant assembly to the hub member, at least a portion of the needle portion of the insertion device being disposed within the lumen defined by the tubular member when the implant assembly is coupled to the hub member.

In some embodiments, the implant assembly includes a tubular member and a sling member, the tubular member having a sidewall defining a lumen, the sidewall defining an opening, the hub member having a projection, the projection being configured to extend through the opening defined by the sidewall of the tubular member to removably couple the implant assembly to the hub member.

In some embodiments, the implant assembly includes a tubular member and a sling member, the tubular member having a sidewall defining a lumen, the sidewall defining an opening, the hub member having a projection, the projection being configured to extend through the opening defined by the sidewall of the tubular member to removably couple the implant assembly to the hub member, at least a portion of the needle portion of the insertion device being disposed within the lumen defined by the tubular member when the implant assembly is coupled to the hub member.

In some embodiments, the implant assembly includes a tubular member and a sling member, the tubular member having a first end portion coupled to the sling member and a second end portion opposite the first end portion, the second end portion having a flared tip.

In some embodiments, the hub member is slideably coupled to the elongate member and is configured to move from a first position with respect to the elongate member to a second position with respect to the elongate member different than the first position.

In some embodiments, the hub member is configured to move from a first position with respect to the elongate member to a second position with respect to the elongate member.

In some embodiments, the implant assembly includes a first tubular member, a second tubular member and a sling member, the sling member having a first end portion coupled to the first tubular member and a second end portion coupled to the second tubular member.

In some embodiments, a method includes inserting a medical device into a body of a patient to a first location within the body of the patient, the medical device including an implant assembly and an insertion device, the insertion device having an elongate member and a hub member, the elongate member having a needle portion and a handle portion, the hub member movably coupled to the handle portion of the elongate member, the hub member being configured to be removably coupled to the implant assembly; moving the hub member from a first position with respect to the elongate member to a second position with respect to the elongate member; and moving the medical device from the first location within the body of the patient to a second location within the body of the patient.

In some embodiments, the moving the hub member from a first position with respect to the elongate member to a second position with respect to the elongate member occurs after the inserting the medical device into a body of a patient to a first location within the body of the patient and before the moving the medical device from the first location within the body of the patient to a second location within the body of the patient.

In some embodiments, the inserting includes inserting the medical device into a pelvic region of the patient.

In some embodiments, the implant assembly includes a tubular member and a sling member, the tubular member defining a lumen, at least a portion of the needle portion of the elongate member being disposed within the lumen when the implant assembly is coupled to the hub member, the needle portion having a tissue piercing tip, the tissue piercing tip being disposed outside of the lumen defined by the tubular member when the hub member is in its first position, the tissue piercing tip being disposed within the lumen when the hub member is disposed in its second position.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this invention. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the disclosure.

What is claimed is:

1. An insertion device, comprising:
an elongate member having a needle portion and a handle portion; and
a hub member movably coupled to the handle portion of the elongate member, the hub member being configured to be removably coupled to an implant assembly.

2. The insertion device of claim 1, wherein the hub member is slideably coupled to the elongate member.

3. The insertion device of claim 1, wherein the needle portion of the elongate member is fixedly coupled to the handle portion of the elongate member.

4. The insertion device of claim 1, wherein the hub member defines a cavity that is configured to receive at least a portion of the implant assembly to frictionally couple the implant assembly to the hub member.

5. The insertion device of claim 1, wherein the hub member includes a projection member, the projection member being configured to extend through a portion of the implant assembly to couple the implant assembly to the hub member.

6. A system, comprising:
an implant assembly; and
an insertion device, the insertion device having an elongate member and a hub member, the elongate member having a needle portion and a handle portion, the hub member movably coupled to the handle portion of the elongate member, the hub member being configured to be removably coupled to the implant assembly.

7. The system of claim 6, wherein the implant assembly includes a tubular member and a sling member, the sling member being coupled to the tubular member.

8. The system of claim 6, wherein the implant assembly includes a tubular member and a sling member, the tubular member defining a lumen, at least a portion of the needle portion of the elongate member being configured to be disposed within the lumen defined by the tubular member when the implant assembly is coupled to the hub member.

9. The system of claim 6, wherein the implant assembly includes a tubular member and a sling member, the tubular member defining a lumen, the hub member defining a cavity, at least a portion of the implant assembly being disposed within the cavity defined by the hub member to couple the implant assembly to the hub member.

10. The system of claim 6, wherein the implant assembly includes a tubular member and a sling member, the tubular member defining a lumen, the hub member defining a cavity, at least a portion of the implant assembly being disposed within the cavity defined by the hub member to couple the implant assembly to the hub member, at least a portion of the needle portion of the insertion device being disposed within the lumen defined by the tubular member when the implant assembly is coupled to the hub member.

11. The system of claim 6, wherein the implant assembly includes a tubular member and a sling member, the tubular member having a sidewall defining a lumen, the sidewall defining an opening, the hub member having a projection, the projection being configured to extend through the opening defined by the sidewall of the tubular member to removably couple the implant assembly to the hub member.

12. The system of claim 6, wherein the implant assembly includes a tubular member and a sling member, the tubular member having a sidewall defining a lumen, the sidewall defining an opening, the hub member having a projection, the projection being configured to extend through the opening defined by the sidewall of the tubular member to removably couple the implant assembly to the hub member, at least a portion of the needle portion of the insertion device being disposed within the lumen defined by the tubular member when the implant assembly is coupled to the hub member.

13. The system of claim 6, wherein the implant assembly includes a tubular member and a sling member, the tubular member having a first end portion coupled to the sling member and a second end portion opposite the first end portion, the second end portion having a flared tip.

14. The system of claim 6, wherein the hub member is slideably coupled to the elongate member and is configured to move from a first position with respect to the elongate member to a second position with respect to the elongate member different than the first position.

15. The system of claim 6, wherein the hub member is configured to move from a first position with respect to the elongate member to a second position with respect to the elongate member.

16. The system of claim 6, wherein the implant assembly includes a first tubular member, a second tubular member and a sling member, the sling member having a first end portion coupled to the first tubular member and a second end portion coupled to the second tubular member.

17. A method, comprising:
inserting a medical device into a body of a patient to a first location within the body of the patient, the medical device including an implant assembly and an insertion device, the insertion device having an elongate member and a hub member, the elongate member having a needle portion and a handle portion, the hub member movably coupled to the handle portion of the elongate member, the hub member being configured to be removably coupled to the implant assembly;
moving the hub member from a first position with respect to the elongate member to a second position with respect to the elongate member; and
moving the medical device from the first location within the body of the patient to a second location within the body of the patient.

18. The method of claim 17, wherein the moving the hub member from a first position with respect to the elongate member to a second position with respect to the elongate member occurs after the inserting the medical device into a body of a patient to a first location within the body of the patient and before the moving the medical device from the first location within the body of the patient to a second location within the body of the patient.

19. The method of claim 17, wherein the inserting includes inserting the medical device into a pelvic region of the patient.

20. The method of claim 17, wherein the implant assembly includes a tubular member and a sling member, the tubular member defining a lumen, at least a portion of the needle portion of the elongate member being disposed within the lumen when the implant assembly is coupled to the hub member, the needle portion having a tissue piercing tip, the tissue piercing tip being disposed outside of the lumen defined by the tubular member when the hub member is in its first position, the tissue piercing tip being disposed within the lumen when the hub member is disposed in its second position.
